(19) United States
(12) Plant Patent Application Publication
(10) Pub. No.: US 2005/0120450 P1
(43) Pub. Date: Jun. 2, 2005

(54) Chrysanthemum plant 'Starlet Sunny'

(50) CHRYSANTHEMUM MORIFOLIUM; CHRYSANTHEMUM

(76) Inventor: Keith G. Lintott, Bognor Regis (GB)

Correspondence Address: Mark K. Dickson;Howrey Simon Arnold & White, LLP, Suites 200 & 300, 2941 Fairview Park Drive, Falls Church, VA, 22042, US

(21) Appl. No.: 10/993,291

(22) Filed: Nov. 18, 2004

Related U.S. Application Data
(60) Provisional application No. 60/525,871, filed on Nov. 28, 2003.

(51) Int. Cl. A01H 5/00

(52) U.S. Cl. PLT/295

(57) ABSTRACT

A new variety of Chrysanthemum plant named ‘Starlet Sunny,’ having a domed, round capitulum of small yellow single type flowers and a good uniform habit and medium habit.

This application claims the benefit of U.S. Provisional Application No. 60/525,871, filed on Nov. 28, 2003.

### LATIN NAME OF THE GENUS AND SPECIES

Botanical classification: Chrysanthemum morifolium.

### VARIETY DENOMINATION

The new Chrysanthemum variety denomination is ‘Starlet Sunny.’

### BACKGROUND OF THE INVENTION

The present invention comprises a new and distinct cultivar of Chrysanthemum botanically known as Chrysanthemum morifolium, and referred to by the cultivar name ‘Starlet Sunny.’

‘Starlet Sunny,’ identified as 30917-0, originated from a cross made in a controlled breeding program in Chichester, West Sussex, United Kingdom. The female parent of the new variety is an undistributed, unpatented seedling identified as ‘G15E2’ and the male parent is the unpatented variety ‘Leyla.’ The new variety ‘Starlet Sunny’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Starlet Sunny’ is a garden type of Chrysanthemum plant variety having a domed, round capitulum of yellow single type flowers.

### Comparison with Female Parent

Plants of the new Chrysanthemum variety ‘Starlet Sunny’ are similar to the parent variety ‘G15E2’ in plant habit and growth rate. However in side-by-side comparisons in Chichester, West Sussex, United Kingdom, under commercial practice, plants of the new Chrysanthemum variety ‘Starlet Sunny’ compared from plants of the parent ‘G15E2’ in the following characteristics:

1. The new variety ‘Starlet Sunny’ produces yellow single type flowers whereas the parent ‘G15E2’ produces purple single type flowers.

2. Plants of the new variety ‘Starlet Sunny’ have smaller and shorter inflorescence than ‘G15E2.’

3. Plants of the new variety ‘Starlet Sunny’ require less natural season days to flower, than ‘G15E2.’

### Comparison with Male Parent

Plants of the new Chrysanthemum variety ‘Starlet Sunny’ are dissimilar to the male parent ‘Leyla’ in plant habit and growth rate. In side-by-side comparisons in Chichester, West Sussex, United Kingdom, under commercial practice, plants of the new Chrysanthemum variety ‘Starlet Sunny’ differed from plants of the parent ‘Leyla’ in the following characteristics.

1. The new variety ‘Starlet Sunny’ has smaller flowers than ‘Leyla.’

2. The new variety ‘Starlet Sunny’ is less vigorous in plant growth than ‘Leyla.’

3. The new variety ‘Starlet Sunny’ requires less natural season days to flower, than ‘Leyla.’

### Comparison with Other Varieties

Plants of the new Chrysanthemum variety ‘Starlet Sunny’ are dissimilar to the unpatented variety ‘Celebrity Yellow’ in plant habitat and growth rate. In side-by-side comparisons in Chichester, West Sussex, United Kingdom, under commercial practice, plants of the new Chrysanthemum variety ‘Starlet Sunny’ differed from ‘Celebrity Yellow’ in the following characteristics:

1. The new variety ‘Starlet Sunny’ produces smaller yellow single type flowers than ‘Celebrity Yellow.’

2. The new variety ‘Starlet Sunny’ is less vigorous in plant habit than ‘Celebrity Yellow.’

3. The new variety ‘Starlet Sunny’ requires less natural season days to flower than ‘Celebrity Yellow.’

### BRIEF DESCRIPTION OF ILLUSTRATIONS

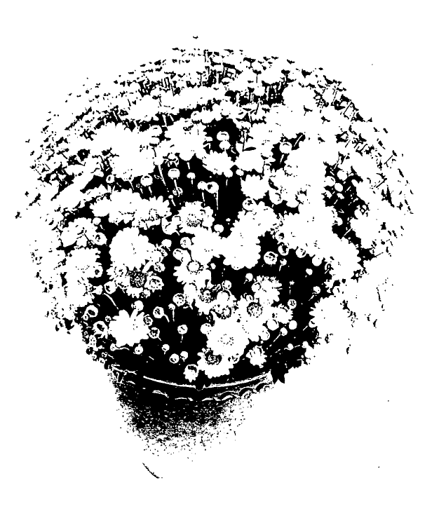

Typical specimens of the plant and flowers for the new Chrysanthemum variety ‘Starlet Sunny’ are shown in the accompanying digital photograph. The colors shown are as true as possible within the usual limits of this kind of illustration. FIG. 1 is a whole plant view of the new variety ‘Starlet Sunny’ grown in a pot. The plant shown in the illustration is 63 days from the commencement of planting outdoors in mid-June.

### DETAILED BOTANICAL DESCRIPTION

The following description of the new Chrysanthemum variety ‘Starlet Sunny’ is of plants grown outside, natural season, in Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a garden Chrysanthemum.

Plants of the new variety have been grown successfully under temperature conditions averaging about 50° F. at night and about 65° F. to 75° F. during the day under natural light conditions. The plants were grown outdoors in mid-June in 4 liter pots using loamless compost. No growth retardants were used. To produce a commercial product the plants may be pinched once with the center bud removed. The typical container size for commercial growth is 4 liter. It has been observed that the shelf life of the new variety is about 25 days with a response time of about 9 weeks natural season. The new variety is suitable for growth in a temperature range of 50° F. to 75° F.

The new variety may be produced as a pot plant. The following description is with respect to a plant produced as a garden pot. In the description of this new Chrysanthemum variety, color values have been taken from The Royal Horticultural Society Colour Chart (R.H.S.C.C.).

### Plant

Plant type: Pot.
Habit: Domed — Round.
Height (cm): 45-50.
Width (cm): 45-50.
Stem:
   Length (cm).—6-9.
   Diameter (cm).—0.5-0.8.
Texture.—Rough.
   Color.—near 137B.
Branching characteristics: Free branching and Spreading.
Lateral branches:
   Length (cm).—12-18.
   Diameter (cm).—0.3-0.5.
   Texture.—Slightly pubescent.
Number of breaks from pinch: 9-11.
Response time: near 63 days natural season.
Vigor: Medium — Medium/Short.
Shelf life: near 25 days.
Disease (susceptibility/resistance observed): None observed to date.
Pest (susceptibility/resistance observed): None observed to date.
Drought and temperature (susceptibility/resistance observed): None observed to date.
Growth retardant type and treatment: No treatment necessary The plants were grown for 9 weeks in natural season conditions.
Propagation:
   Type.—Vegetative Propagation Via Stem Cuttings.
   Time to rooting.—12 days with soil temperatures of 70° F.
   Rooting habit.—Fine and Fibrous.

### Foliage

Number of leaves per lateral branch: 11-14.
Compound or single: Single.
Arrangement of leaves: Alternate.
   Shape of leaf.—Typically 5 lobed ovate.
   Size of leaf.—Width (cm): 2.5-3 Length (cm): 3.0-4.5.
   Leaf apex.—Acute.
   Base.—Cordate.
   Attachment.—petioled.
   Aspect.—Slightly Undulating.
   Margin.—Palmately Lobed.
   Surface characteristics.—Top: Slightly pubescent Bottom: Slightly pubescent.
Petiole:
   Color.—near 138B.
   Length (cm).—1.5-2.0.
   Diameter (cm).—0.2-0.4.
Venation: net, mid vein prominent at underside.
   Color.—upper side: near 144B under side: near 144B.
Color:
   Mature leaf, upper side.—near 138A; under side: near 138B.
   Young leaf, upper side.—near 138A; under side: near 138B.

### Flower

Flower appearance: Matte.
Flower type: Single.
Flower form: Flat—Slightly Cupped.
Flower shape: Round.
Flowering habit: Cyme.
Number of blossoms per branch: 11-20.
Typical and observed flowering season: Natural season months August/September.
Inflorescence form: Corymbiform.
   Depth of fully expanded blossoms (cm).—1.
   Diameter of fully expanded blossoms (cm).—2.5-3.0.
Phyllaries:
   Number.—23-25.
   Color.—near 137C.
   Length (cm).—0.4-0.7.
   Width (cm).—0.1-0.2.
   Texture/Appearance.—Shiny — Slightly pubescent.
Peduncle:
   Length (cm).—11-19.
   Diameter (cm).—0.3-0.6.
   Angle from stem (degrees).—20°-25°.
   Color.—near 137C.
   Surface.—Slightly pubescent.
   Habit.—Slightly curving.
   Strength.—Medium.
Pedicel:
   Length.—terminal (cm): 5-6; lateral (cm): 9-11.
   Diameter (cm).—0.2-0.4.
   Color.—near 137C.
   Surface.—Slightly pubescent.
   Habit.—Slightly curving.
   Strength.—Medium.
Ray florets:
   Form/Shape.—Straight.
   Texture/Appearance.—Matte.
   Number per flower.—28-35.
   Length (cm).—1.5-1.8; Width (cm): 0.4-0.5.
   Apex.—Rounded.
   Base.—Tubular.
   Margin.—Entire.
Disc florets:
   Form/Shape.—Cylindrical.
   Texture/Appearance.—Shiny.
   Number per flower.—120-150.
   Length (cm).—0.5-0.7; Width (cm): <0.1-0.1.
   Diameter of disc (cm).—0.7-1.0.
Fragrance: Slight.
Flower bud (at onset of color):
   Length (cm).—0.8.
   Diameter (cm).—0.8.
   Form/Shape.—Globular.
General flower color:
   1. Ray florets, upper side.—Immature: near 6A Mature: near 6C Older/Fading: near 6D.
   2. Ray florets, under side.—Immature: near 6A Mature: near 6C Older/Fading: near 6D.
   3. Disc florets.—Immature: near 13A Mature: near 13B Older/Fading: near 13B.
   4. Bud.—near 137B.
Flower progression with age: Flowers fade slightly and uniformly with age.

### Reproductive Organs

Gynoecium present on ray and disc florets.
Ray florets per individual flower: Pistillate.
   Pistil number.—28-35 observed.
   Stigma color.—near 144D.
   Stigma shape.—Forked.
   Style color.—near 144D.
   Style length (cm).—0.1-0.2.
Disc florets per individual flower: Pistillate.
   Pistil number.—45-60 observed.
   Stigma color.—near 144D.
   Stigma shape.—Forked.
   Style color.—near 144D.
   Style length (cm).—0.2-0.3.
Androecium present on disc florets only.
Disc florets per individual flower:
Staminate:
   Stamen number.—50-60 observed.
   Anther shape.—Ovoid.
   Anther color.—near 14C.
   Pollen color.—near 12A.
   Pollen quantity.—Slight.
Fruit and seeds: None observed.

1. A new and distinct variety of Chrysanthemum plant, substantially as described and illustrated herein.
